(19) United States
(12) Patent Application Publication
(10) Pub. No.: US 2005/0196445 A1
(43) Pub. Date: Sep. 8, 2005

(54) Edible microcrystalline cellulose and carrageenan coating composition

(76) Inventor: Michael Augello, Marlboro, NJ (US)

Correspondence Address: Patent Administrator, FMC Corporation, 1735 Market Street, Philadelphia, PA, 19103, US

(21) Appl. No.: 11/070,615

(22) Filed: Mar. 2, 2005

Related U.S. Application Data
(60) Provisional application No. 60/549,785, filed on Mar. 3, 2004.

(51) Int. Cl. A61K 9/24; A61K 9/36; A61K 31/716; A61K 31/737

(52) U.S. Cl. 424/472; 514/54; 514/57

(57) ABSTRACT

The present invention relates to novel, edible, hardenable, prompt release coatings for pharmaceutical, nutraceutical and veterinary solid dosage forms, confectionery, seeds, animal feed, fertilizers, pesticide tablets, and foods, etc., comprising microcrystalline cellulose and a film forming amount of carrageenan, wherein such coatings do not contain a plasticizer, a strengthening polymer or a surfactant.

This application claims the benefit of U.S. Provisional Application No. 60/549,785, filed Mar. 3, 2004.

### FIELD OF THE INVENTION

The present invention relates to novel, edible, prompt release coating compositions for pharmaceutical tablets, confectionery, seeds, animal feed, fertilizers, pesticide tablets, tableted veterinary medications, and foods, etc., comprising microcrystalline cellulose and a film forming amount of carrageenan, wherein such coating compositions do not contain a plasticizer, a strengthening polymer or a surfactant.

### BACKGROUND OF THE INVENTION

It is a common practice to coat pharmaceutical and veterinary tablets to obtain several advantages. Among these are to mask unpleasant tasting active ingredients with a barrier coat, to improve the surface characteristics of tablets to make them easier to swallow, to reduce the absorption of water which can potentially degrade the active ingredient or promote some other undesirable change in the tablet structure, and simply to make a more elegant appearing tablet.

Another very important function of a pharmaceutical or veterinary tablet coating is to improve the integrity of the tablet itself. Uncoated tablets are often subject to being abraded or chipped, causing a loss of active ingredient in the process. More dramatically, they may be broken into two or more pieces. One measure of a useful coating is its ability to prevent any of these physical degradations of tablet structure. The effectiveness of a coating material to prevent abrading, chipping, or breakage of the tablet is determined by friability testing.

Confectionery and foods may be coated with a formulation which will preserve the confection or food from deteriorating by contact with the air and the humidity in the atmosphere. Coatings also can provide improved appearance and desirable organoleptic properties to the food as well as preventing loss of flavor.

Seeds may be coated with a coating to preserve the viability of the seeds by protecting against moisture. Alternatively, a dye can be included in the coating formulation to identify the seeds as to quality, type, or some other designation. Frequently, a pesticide, e.g., a fungicide, is incorporated into the coating formulation to protect both the seed itself and the seedling that results from germination of the seed. In all cases, this coating must not decrease the viability of the seeds or interfere with germination when the seeds are planted in the soil.

Animal feed may be coated with formulations of this invention which have been prepared by including vitamins, hormones, antibiotics, or the like to benefit the livestock which will consume the feed.

Fertilizers, in either granular or tableted forms, may be coated to retain the integrity of the form and, especially, to protect the fertilizer from moisture which can cause agglomeration during storage, and make rapid, even application to the soil difficult or inconvenient.

Coating of tableted pesticide formulations serve to maintain the integrity of the tablets until they are placed in water where they rapidly disintegrate, forming a solution or slurry to be applied to the soil of plants. A second, and equally important, function of the coatings on tablets containing pesticides is to prevent human contact with the pesticide, thereby increasing safety of those handling and applying the pesticide.

Currently, most commercially available edible coatings utilize hydroxypropylmethyl cellulose (HPMC) as the polymer for useful coatings. Other synthetic film-formers that are commonly used include ethylcellulose, methylcellulose, polyvinylpyrrolidone, and polydextrose. These coating materials may be used alone or in combination with secondary film-formers such as sodium alginate or propylene glycol alginate. See for example, U.S. Pat. Nos. 4,543,370, 4,802,924, and 4,513,019. All of these materials, alone or in combination, are film-formers which make them particularly suitable as the basic tablet coating material. They are usually used in combination with other ingredients including fillers, e.g., titanium dioxide or talc, plasticizers, such as high molecular weight polyethylene glycols, dibutyl sebacate, and triethyl citrate, surfactants, and often coloring materials such as a food dye or pigment.

In the preparation of a coating formulation to be sprayed, the polymer is usually dissolved or dispersed in water along with the other ingredients of the formulation. Since many polymers require significant :time to become fully hydrated, the coating formulation must be prepared in advance of the time it is to be applied to the tablets. A common procedure is to prepare these coating formulations the day preceding the coating operation.

In addition, coatings based on HPMC may harden and therefore increase tablet disintegration times. An increase in disintegration time delays the bioavailability of the active ingredient at least in proportion to the increase in disintegration time.

Other coatings described in the art include microcrystalline cellulose, carrageenan and at least one of a strengthening polymer, plasticizer or surface active agent. For example, see U.S. Pat. No. 6,432,448.

The coatings of this invention meet U.S. Pharmacopoeia standards for rapid or immediate dissolution (U.S.P. monograph 23) of active ingredients from tablets or other solid dosage forms coated with them. They provide prompt release or dissolution consistent with the release rates which is normally obtained with the uncoated tablets or other substrates. Thus, they do not adversely impact or retard release of active ingredients from a substrate coated with them. Further, the coatings of this invention are readily dispersed and rapidly hydrated in aqueous media for application to a coating substrate, and provide elegant coatings which have all the benefits of coatings now in commercial use without the drawbacks that are common to them.

### SUMMARY OF THE INVENTION

The present invention relates to novel, edible, hardenable, prompt release coatings for pharmaceutical, veterinary and nutraceutical solid dosage forms, confectionery, seeds, animal feed, fertilizers, pesticide tablets, tableted veterinary medications, and foods, etc., comprising microcrystalline cellulose and a film forming amount of carrageenan, wherein such coatings do not contain a plasticizer, a strengthening polymer or a surfactant. All components of the formulation are typically pharmaceutically acceptable food grade materials.

In this application the term carrageenan is to be understood as meaning any naturally derived carrageenan, including the grades further defined below as iota, kappa, and lambda carrageenan. The terms MCC or microcrystalline cellulose are to be understood as meaning hydrolyzed cellulose, including grades which are not attrited as well as those that are attrited, microreticulated cellulose. These definitions are intended to apply throughout this application unless-a contrary meaning is clearly indicated.

The present invention is also directed to solid dosage and other forms coated with these coatings as well as to methods of coating such solid dosage and other forms.

### DETAILED DESCRIPTION OF THE FIGURES

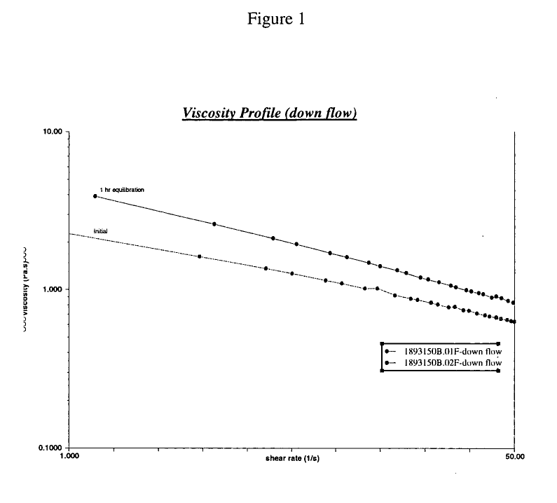

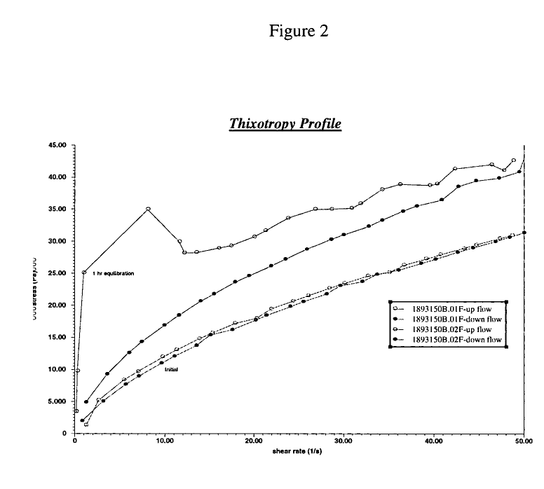

FIGS. 1 and 2 show the Brookfield viscosity and rheological evaluations for the dispersion prepared in Example 4.

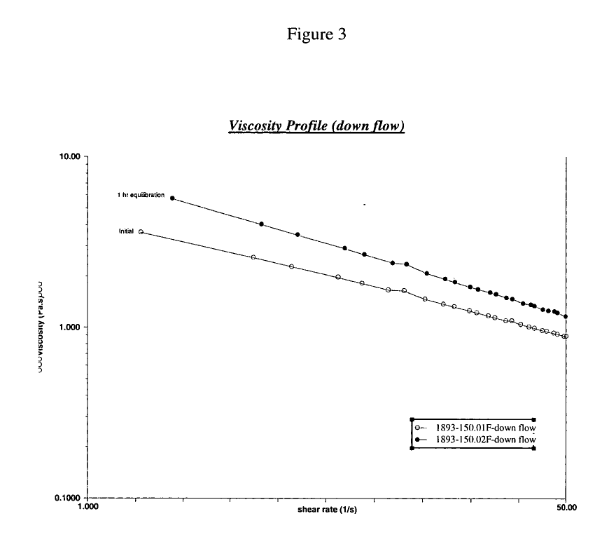

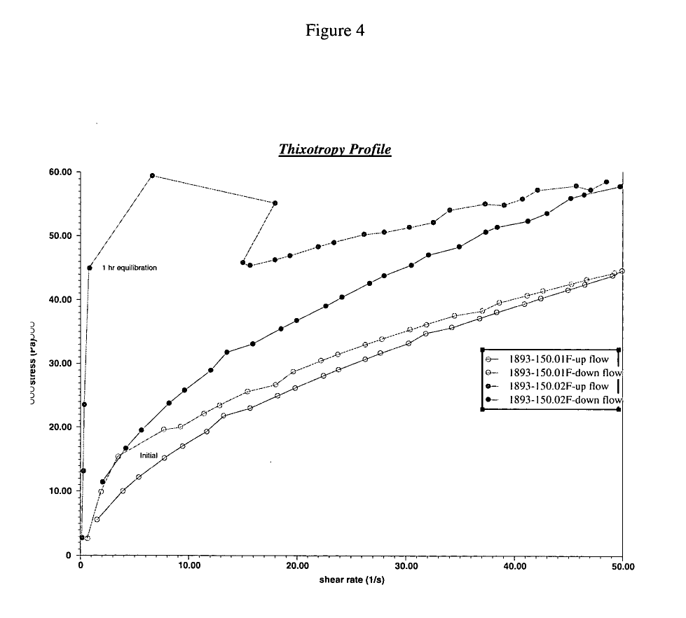

FIGS. 3 and 4 show the Brookfield viscosity and rheological evaluations for the dispersion prepared in Example 6.

### DETAILED DESCRIPTION OF THE INVENTION

For purposes of this application, the term “edible” is intended to mean food grade materials that are approved by regulatory authorities for use in pharmaceutical or food applications. The term “hardenable” used to describe the coating compositions of this invention is intended to include only those coating compositions that are capable of being dried from an aqueous solution or dispersion thereof into a solid coating which resists abrasive forces, i.e. a hardened coating, as distinguished from those “enrobing” coatings on confections which set up into a soft coating that can be handled and packaged but which do not resist abrasive forces significantly. The terms “immediate”, “rapid” or “prompt” release as applied to dissolution rates or times for the coating compositions of this invention or tablets coated with the compositions of this invention means that the coatings of this invention meet U.S. Pharmacopoeia standards (U.S.P. monograph 23) for rapid or immediate dissolution of active ingredients from tablets or other solid dosage forms coated therewith. Thus, they provide prompt release or dissolution consistent with the release rate that is normally obtained with the uncoated tablets or other substrate. They do not, consistent with the pharmacopeia standards above, when placed in aqueous media or ingested by, e.g., a human, significantly impact or retard release or dissolution of tablets or other solid dosage forms coated therewith. For example, coatings made in accordance with the present invention are substantially or completely disintegrated and/or dissolved within less than 10 minutes after being ingested or placed in aqueous media. Thus, when a pharmaceutical solid dosage form is coated with the coating of this invention and ingested by a human or other animal, the coating of this invention is dissolved or disintegrated prior to leaving the stomach. These definitions are intended to apply throughout this application unless a contrary meaning is clearly indicated.

Microcrystalline cellulose is a purified, partially depolymerized, cellulose that is produced by treating a source of cellulose, preferably, alpha cellulose, in the form of a pulp from fibrous plants, with a mineral acid, preferably, hydrochloric acid. The acid selectively attacks the less ordered regions of the cellulose polymer chain, thereby exposing and freeing the crystallite sites, forming the crystallite aggregates that constitute microcrystalline cellulose. These are then separated from the reaction mixture and washed to remove degraded by-products. The resulting wet mass, generally containing 40 to 60 percent moisture, is referred to in the art by several names, including hydrolyzed cellulose, microcrystalline cellulose, microcrystalline cellulose wetcake, or simply wetcake. It is this hydrolyzed cellulose, which may be further modified, for example, by attrition or spray drying, that is utilized in accordance with the present invention.

Microcrystalline cellulose may also be produced by a process known as steam explosion. In this process wood chips are placed in a chamber into which super-heated steam is introduced. After being maintained for period of about 1-5 minutes, the exit valve is opened rapidly, releasing the contents explosively and yielding microcrystalline cellulose. Although no additional acid is introduced into the reaction mixture, the acidic materials in the wood chips and the elevated temperature and pressure hydrolyze the cellulose and degrade it.

The microcrystalline cellulose and carrageenan may be either coprocessed or mixed in a dry blend.

The weight ratio of microcrystalline cellulose to carrageenan in the compositions of this invention may vary depending on the application, but generally range from about 90:10 to about 65:35, preferably from about 85:15 to about 70:30, more preferably, approximately 75:25. A particular advantage for the dry, physical 10 blends is that the ratio can be easily changed by simple blending techniques rather than manufacturing different ratios of coprocessed material. Thus, the dry, physical blends provide significantly greater flexibility for specific applications having different requirements. Pharmaceutical and veterinary solid dosage forms containing certain active ingredients may require increased carrageenan content in the composition to ideally coat the tablets.

Examples of carrageenan to be used in this invention include iota, kappa and lambda carrageenan. The preferred type of carrageenan, a polysaccharide which is comprised of repeating galactose units and 3,6-anhydrogalactose units, that is suitable for the coprocessed compositions of this invention is referred to as iota carrageenan. A rich source of iota carrageenan is the seaweed Eucheuma spinosum. Iota carrageenan to be used in the present invention may be purchased from FMC Corporation, Philadelphia, Pa. The approximate content of anhydrogalactose units in iota carrageenan is 30% whereas kappa carrageenan has 34% anhydrogalactose units and lambda carrageenan is essentially devoid of these units. Carrageenans are also characterized by the amount of ester sulfate groups that are present on both the galactose and anhydrogalactose units. The ester sulfate content of iota carrageenan may range from about 25% to 34%, preferably about 32%. This is intermediate between kappa carrageenan which has a 25% ester sulfate content and lambda carrageenan which has a 35% ester sulfate content. The sodium salt of iota carrageenan is soluble in water, but different grades of iota carrageenan require heating water to different temperatures to dissolve them. The iota carrageenans that are suitable for the coprocessed MCC/iota carrageenan material of this invention are soluble in water heated up to 80° C. (176° F.). Preferred grades of iota carrageenan are soluble at lower temperatures, for example, at 50° C. (122° F.).

A physical blend of microcrystalline cellulose (e.g., Avicel PH-105, available from FMC Corporation, average particle size 20 microns) and a film-forming amount of carrageenan, e.g., iota carrageenan, has been found to provide desirable coating composition attributes.

It is preferred that the average particle size of the microcrystalline cellulose used in a dry blend with the carrageenan should be below 100 microns, advantageously below about 50 microns, preferably in the range of about 1-50 microns, more preferably, about 1-30 microns. Elegant, high performance coating formulations within the scope of this invention may be prepared from such dry, physical blends of microcrystalline cellulose and carrageenan.

Edible coating formulations of this invention are prepared according to a simple procedure. Preparation of a dry mixture comprised of coprocessed microcrystalline cellulose/carrageenan or a dry blend of microcrystalline cellulose and the carrageenan precedes the hydration step required to prepare the final coating formulation. This dry mixture is then added slowly to a vortex of stirred, purified water. Stirring of this mixture is continued for a sufficient period to allow all of the components to be fully hydrated. A simple propeller mixer provides adequate agitation for rapid hydration.

The period of hydration may be as short as 0.5 hour. It may, and preferably should, be longer, but more than 3 hours is not believed to be necessary. Hydration can take place at room temperature or at elevated temperatures as high as 65.5° C. (150° F.), preferably, at a temperature about 48.9° C. (120° F.). The time required for full hydration and the viscosity of the dispersion are both considerably reduced when the dispersion is prepared at an elevated temperature, but coating dispersions prepared at ambient temperature only require an increase in hydration time and a slight reduction in solids content to perform completely satisfactorily. As previously stated, these formulations may be prepared on the day preceding the coating operation, if that is more convenient; however, a period of mixing will be required to overcome the thixotropic behavior of a formulation which sets up during overnight storage.

An example of microcrystalline cellulose in the present invention is: Avicel PH-105 available from FMC Corporation (Philadelphia, Pa.); and an example of iota carrageenan is A-Vis-S, also available from FMC Corporation. In one particular embodiment, the microcrystalline cellulose and carrageenan are present in a ratio of 75:25. These materials within any ratio of the present invention can be charged into any suitable size V-blender, mixing for a suitable time based on the batch size and discharging into clean poly-lined containers, awaiting release.

The following optional ingredients may be used in coating formulations based on either the coprocessed microcrystalline cellulose/carrageenan or a blend of microcrystalline cellulose and a carrageenan.

Fillers may include, for example, talc, titanium dioxide, calcium carbonate, dicalcium phosphate and carbohydrates, e.g. starch, maltodextrin, lactose, and other sugars. Of these, maltodextrin is preferred filler. Edible coloring agents and opacifiers such as talc, titanium dioxide, food dyes or lakes may be added. Anti-tack agents may also be present in the composition.

A coating formulation of this invention may be sold as a ready-to-use dispersion in water, provided it has been prepared under aseptic condition. Heating the water to an elevated temperature, for example, 85° C., prior to preparation of the dispersion has shown that bacteria, mold, and yeast growth are prevented for at least 48 hours on agar pour plates. Therefore, if the containers for the dispersion are properly sanitized and then kept closed after being filled until they are used, there is little likelihood of bacteria, mold, or yeast growing in the dispersion. Alternatively, if a formulation is to be sold as an aqueous dispersion to be stored for a period of time a preservative may be added. A combination of methyl paraben and propyl paraben has been found to be useful in this regard.

The viscosity of the hydrated formulation is a limiting factor. It must be low enough to be pumped to a spray unit continuously and then sprayed evenly in a useful pattern onto the tablets being coated. A useful concentration of the dry ingredients in water on a weight percentage basis, therefore, is about 6.5% to about 11%, preferably about 8.0% to about 11%. To assure uniformity of the coating composition, it is preferable to maintain agitation of the aqueous dispersion during the entire period of its being sprayed onto the solid forms such as solid dose forms, confectionery, seeds, animal feed, fertilizer, pesticide tablets, tableted veterinary medications, or food.

Any commercial spray coater may be used to apply the coating to the tablets. Examples of useful coaters are Vector High Coaters manufactured by Vector Corporation and Accela-Cota manufactured by Thomas Engineering. Equipment variables which one skilled in the art can manipulate to provide an elegant coating based on the microcrystalline cellulose/carrageenan materials, either coprocessed or blended, include inlet temperature, outlet temperature, air flow, speed of rotation of the coating pan, and the rate at which the coating formulation is pumped to the coater. It is important that the inlet and outlet temperatures be controlled so that they are high enough to efficiently dry the coating to prevent the tumbling action of the already-coated dose forms from damaging the newly-applied coating before more coating is applied to the same dose forms.

The level of coating applied to pharmaceutical, nutraceutical or veterinary solid dosage forms is preferably between about 0.5 weight % and about 4 weight %, more preferably about 2% by weight to about 3.5% by weight, based on the weight of the uncoated dosage forms. This level of coating will provide an elegant, serviceable coating to a wide variety of dosage forms. To apply a heavier coating to dosage forms would not be economical, and it might adversely affect disintegration of the dosage forms or other properties. Too light a coating would not provide optimal properties normally expected from a dosage form coating, e.g., improved friability or adequate taste masking.

For confections the coating level should be about 5% to about 10% by weight of the uncoated confection. Seed coatings should be in the range of about 3% to about 6% by weight of the uncoated seeds. Fertilizers and pesticide tablets benefit from coating of 1% to about 3%, by weight of the uncoated granules or tablets.

The following examples in which percentages are weight percent and tablet hardness is in Kiloponds (Kp) are provided to demonstrate the method of preparation and application of these elegant coatings, but they are not intended to be limiting as to amounts and the type of optional ingredients or the specific method of application of the dosage form coating described herein. Unless otherwise indicated, all parts, percentages, etc. are by weight.

### EXAMPLE 1

### Aqueous Film Coating of the Present Invention 500 mg Acetaminophen Caplets

Microcrystalline cellulose and iota carrageenan were blended together and tested as follows.

| | | |
|---|---|---|
| FMC | Avicel PH-105 | 75% |
| FMC | A-Vis-S (11 cps) | 25% |
| | | 100% |

Rheology (Brookfield) - Percent Solids 10%
Mixer Speed - 600 RPM, Mixed for 1 Hour

| | Cps | pH |
|---|---|---|
| T⁰ | 900 | 6.46 |
| T²⁴ | 2500 | 6.70 |
| T²⁴ Reshear | 1250 | |

Aqueous Film Coating Procedure

| Pan | Vector LDCS, 15″ Coating Pan | |
|---|---|---|
| Spray Apparatus | # 1 Gun, Jet Spray | |
| | 1.0 mm Fluid Nozzle, 134255 Air Cap | |
| Delivery System | Masterflex Pump, Model Digital Console Drive | |
| | Model #7523-50, Fitted with #1 Pump Head, #16 | |
| | Tubing/94600 | |
| Substrate | Acetaminophen - 500 mg Caplets | |
| Weight Gain | 3% | |
| Charge | 1.5 kg | |
| Coating Parameters | Inlet Air Temp, ° C. | 75-87 |
| | Exhaust Air Temp, ° C. | 31-36 |
| | CFM | 40 |
| | ATM (psi) | 25 |
| | Pan Speed (RPM) | 11-14 |
| | Delivery Rate (gms/ml) | 13-19 |

Physical Testing - Average/10 Caplets

| | Film-Coated Caplets | Cores |
|---|---|---|
| Ten Caplet Weight (gms) | 5.72 | 5.55 |
| Thickness (mm) | 6.14 | 6.07 |
| Hardness (kp) | 12.2 (initial) | 7-8 |
| | 12.2 (after 24 hours) | |
| Disintegration, | <5 minutes | <2 minutes |
| 37° C. DI Water | | |
| Friability, 30 minutes | 1 hr out of pan - 0.0184% | 4.06% |
| | 3 hrs out of pan - 0.031% | |
| | 24 hrs out of pan - 0.09% | |

Chemical Analysis

| Film-Coated Caplets | MCC/CGN |
|---|---|
| Dissolution Profile | Lot # G1893-149 |
| 10 min | 76 ± 10.3 |
| 20 min | 95 ± 4.1 |
| 30 min | 100 ± 2.4 |

### EXAMPLE 2

### Aqueous Film Coating of Present Invention 500 mg Acetaminophen Caplets

The following microcrystalline cellulose/carrageenan blend was prepared and tested.

| | | |
|---|---|---|
| FMC | Avicel PH-105 | 75% |
| FMC | A-Vis-S (12 cps) | 25% |
| | | 100% |

Rheology (Brookfield) - Percent Solids 10%
Mixer Speed - 600 RPM, Mixed for 1 Hour

| | Cps | pH |
|---|---|---|
| T⁰ | 600 | 6.72 |
| T²⁴ | 4350 | 6.84 |
| T²⁴ Reshear | 2500 | |

Aqueous Film Coating Procedure

| Pan | Accela Comp-U-Coat | |
|---|---|---|
| Spray Apparatus | # 2 Binks Guns | |
| | 1.0 mm Fluid Nozzle, 40100 Air Cap | |
| Delivery System | Masterflex Pump, Model Digital Console Drive | |
| | Model #7523-50, Fitted with #1 Pump Head, #24 | |
| | Tubing 94600 | |
| Substrate | Acetaminophen - 500 mg Caplets | |
| Weight Gain | 3% | |
| Charge | 12.0 kg | |
| Coating Parameters | Inlet Air Temp, ° C. | 55-75 |
| | Exhaust Air Temp, ° C. | 36-39 |
| | CFM | 255-260 |
| | ATM (psi) | 25 |
| | Pan Speed (RPM) | 11.0 |
| | Delivery Rate (gms/ml) | 35-70 |

Physical Testing - Average/10 Caplets

| | Film-Coated Caplets | Cores |
|---|---|---|
| Ten Caplet Weight (gms) | 5.71 | 5.55 |
| Thickness (mm) | 6.09 | 6.07 |
| Hardness (kp) | 11.1 (initial) | 7-8 |
| | 10.5 (after 24 hours) | |
| Disintegration, | <5 minutes | <2 minutes |
| 37° C. DI Water | | |
| Friability, 30 minutes | 1 hr out of pan - 0.067% | 4.06% |
| | 3 hrs out of pan - 0.124% | |
| | 24 hrs out of pan - 0-0.061% | |

Chemical Analysis

| Film-Coated Caplets | MCC/CGN |
|---|---|
| Dissolution Profile | |
| 10 min | 69 ± 10.9 |
| 20 min | 95 ± 3.2 |
| 30 min | 99 ± 0.8 |

Technical Film Properties

| Stress at Break | Strain at Break | Toughness |
|---|---|---|
| (mpa) | (%) | (mpa) |
| 7 ± 3 | 7 ± 8 | 0.3 ± 0.3 |

### EXAMPLE 3

### Aqueous Film Coating of the Present Invention 600 mg Calcium Carbonate Caplets

The following blend of microcrystalline cellulose and iota carrageenan was prepared and tested.

| | | |
|---|---|---|
| FMC | Avicel PH-105 | 75% |
| FMC | A-Vis-S (12 cps) | 25% |
| | | 100% |

Rheology (Brookfield) - Percent Solids - 10%
Mixer Speed - 600 RPM, Mixed for 1 Hour

| | cps | pH |
|---|---|---|
| T⁰ | 800 | 6.72 |
| T²⁴ | N/A | N/A |
| T²⁴ Reshear | N/A | |

Aqueous Film Coating Procedure

| | |
|---|---|
| Pan | Accela Comp-U-Coat, 24″ Pan |
| Spray Apparatus | #2 Binks Guns |
| | 1.0 mm Fluid Nozzle, 40100 Air Cap |
| Delivery System | Masterflex Pump, Model Digital Console Drive |
| | Model #7523-50, Fitted with #1 Pump Head, |
| | #24 Tubing 94600 |
| Substrate | Calcium Carbonate - 600 mg Caplets/Vitamin D |
| Weight Gain | 3% |
| Charge | 12.0 kg |

| | | |
|---|---|---|
| Coating Parameters | Inlet Air Temp, ° C. | 55-70 |
| | Exhaust Air Temp, ° C. | 33-41 |
| | CFM | 258-263 |
| | ATM (psi) | 25 |
| | Pan Speed (RPM) | 11-13 |
| | Delivery Rate (gms/ml) | 30-80 |

Physical Testing - Average/10 Caplets

| | Film-Coated Caplets | Cores |
|---|---|---|
| Ten Caplet Weight (gms) | 1.601 | 1.555 |
| Thickness (mm) | 7.12 | 6.89 |
| Hardness (kp) | 30.9 (initial) | 33.9 |
| | 27.7 (after 72 hours) | |
| Disintegration, 37° C. DI | <8 minutes | <5 minutes |
| Water | | |
| Friability, 30 minutes | 1 hr out of pan - 0.025% | 1.33% |
| | 3 hrs out of pan - 0.006% | |
| | 72 hrs out of pan - 0.003% | |

### EXAMPLE 4

### Rheological Evaluation

The sample below was tested at 10% solids. 100 g of sample was dispersed in a 1500 ml glass beaker containing 900 g of deionized water using a Caframo mixer (medium blade) set at 600 rpm. After all the material was added to the water, the speed of the mixer was increased to 1000 rpm and mixed for one hour. After hydration was completed, the dispersion was evaluated for Brookfield viscosity followed by further rheological evaluation using the TA instrument. The dispersion was then placed in the 20° C. water bath for the duration of the test (overnight).

| Ingredient | Mfg | % |
|---|---|---|
| Avicel PH-105 | FMC | 75 |
| A-Vis-S (11 cps) | FMC | 25 |

The sample was evaluated using the TA Instruments, AR-1000N Rheometer according to the following parameters:

A 6 cm flat acrylic plate with a solvent trap, 20° C. temperature and 500 μm gap was the geometry used for all measurements. The sample was analyzed using a step ramp, 0-50-0 sec⁻¹ in a linear mode. The measurement was taken within five minutes after the dispersion was complete. The measurement was repeated after one hour equilibration on the peltier plate.

The Brookfield, RVT viscometer was used to measure the viscosity of the dispersion at time 0 (immediately after dispersion), 1, 3, 5 and 24 hrs. Following the 24 hrs measurement, the sample was stirred for five minutes and the viscosity was measured immediately. The following parameters were used for each measurement: spindle #4 at 20 rpm for 20 seconds.

The viscosity of the dispersion increased from 700 cps to 3300 cps in 24 hours and decreased to 1250 cps after re-sheared. The pH remained stable after 24 hours. The sample displayed an increase in viscosity and thixotropy after one hour equilibration on the peltier.

Brookfield Viscosity

| | Viscosity (η) (mPa.s) | | | | | | |
|---|---|---|---|---|---|---|---|
| 10% solids | T₀ | T₁ | T₃ | T₅ | T₂₄ | T_{24*} | pH/pH 24 hr |
| a. | 700 | 1500 | 2550 | 2800 | 3300 | 1250 | 6.05/6.03 |

T_{24*} sample was resheared for 5 minutes

See FIGS. 1 and 2.

### EXAMPLE 5

### 9% Solids Concentration of Various A-Vis-S Viscosities (11-15 cps).

A-Vis-S (11 cps)

| | cps | PH |
|---|---|---|
| T⁰ | 400 | 6.71 |
| T²⁴ | 700 | 6.63 |
| T²⁴ Reshear | 400 | |

A-Vis-S (12 cps)

| | cps | pH |
|---|---|---|
| T⁰ | 700 | 6.74 |
| T²⁴ | 1950 | 6.75 |
| T²⁴ Reshear | 900 | |

A-Vis-S (13 cps)

| | cps | pH |
|---|---|---|
| T⁰ | 950 | 6.86 |
| T²⁴ | 1950 | 6.98 |
| T²⁴ Reshear | 1100 | |

A-Vis-S (14 cps)

| | cps | PH |
|---|---|---|
| T⁰ | 1100 | 7.06 |
| T²⁴ | 1900 | 6.71 |
| T²⁴ Reshear | 950 | |

A-Vis-S (15 cps)

| | cps | PH |
|---|---|---|
| T⁰ | 1300 | 7.23 |
| T²⁴ | 3000 | 7.01 |
| T²⁴ Reshear | 2100 | |

### EXAMPLE 6

The sample below was tested at 10% solids.

100 g of sample was dispersed in a 1500 ml glass beaker containing 900 g of DI water using a Caframo mixer (medium blade) set at 600 rpm. After all the material was added to the water, the speed of the mixer was increased to 1000 rpm and mixed for one hour. After hydration was completed, the dispersion was evaluated for Brookfield viscosity followed by further Theological evaluation using the TA instrument. The dispersion was then placed in the 20° C. water bath for the duration of the test (overnight).

Sample:

| Ingredient | Mfg | % |
|---|---|---|
| Avicel PH-105 | FMC | 75 |
| A-Vis-S (12cps) | FMC | 25 |

The sample was evaluated using the TA Instruments, AR-1000N Rheometer according to the following parameters:

A 6 cm flat acrylic plate with a solvent trap, 20° C. temperature and 500 μm gap was the geometry used for all measurements. The sample was analyzed using a step ramp, 0-50-0 sec⁻¹ in a linear mode. The measurement was taken within five minutes after the dispersion was complete. The measurement was repeated after one hour equilibration on the peltier plate.

Brookfield Viscosity

The Brookfield, RVT viscometer was used to measure the viscosity of the dispersion at time 0 (immediately after dispersion), 1, 3, 5 and 24 hrs. Following the 24 hrs measurement, the sample was stirred for five minutes and the viscosity was measured immediately. The following parameters were used for each measurement: spindle #4 at 20 rpm for 20 seconds.

The viscosity of the dispersion increased from 2200 cps to 5 100 cps in 24 hours and decreased to 2300 cps after re-sheared. The pH remained stable after 24 hours. The sample displayed an increase in viscosity and thixotropy after one hour equilibration on the peltier.

Brookfield Viscosity

| | Viscosity (η) (mPa.s) | | | | | | |
|---|---|---|---|---|---|---|---|
| 10% solids | T₀ | T₁ | T₃ | T₅ | T₂₄ | T_{24*} | pH/pH 24 hr |
| b. | 2200 | 4900 | 5000 | 5000 | 5100 | 2300 | 6.35/6.34 |

T_{24*} sample was resheared for 5 minutes

See FIGS. 3 and 4

### EXAMPLE 7

### Dissolution Testing of Acetaminophen Caplets

Samples of two coated caplets below satisfied the USP 26 stage 1 dissolution requirement for acetaminophen tablets of a release from each unit of not less than 80+5% of the labeled amount of acetaminophen in 30 minutes. All coatings ruptured within 90 seconds of testing.

USP apparatus 2 (paddle), 50 rpm, 900 mL, 0.05 M phosphate buffer, pH 5.8° was used.

### Analysis (UV, 243 nm)

#1: Acetaminophen caplet cores (Advance Pharmaceutical, 01F015, 500-mg (claim weight) coated with 3% of the following coating: 75.0% Avicel® PH-105 MCC and 25.0% A-VIS-S iota carrageenan (11 cps).

#2: Same as #1 but with 25% A-VIS-S iota carrageenan (11 cp) scale-up from 1.5 to 12 kg.

Dissolution Testing of Coated
Acetaminophen Caplets of the Present Invention

| | Mean Percent Release ± S. D. (n = 6) | |
|---|---|---|
| Time | | |
| (Min) | #1 | #2 |
| 10 | 76 ± 10.3 | 69 ± 10.9 |
| 20 | 95 ± 4.1 | 95 ± 3.2 |
| 30 | 100 ± 2.4 | 99 ± 0.8 |

1. An edible, hardenable, prompt release coating composition comprising microcrystalline cellulose and a film forming amount of carrageenan, wherein said coating composition does not contain a plasticizer, strengthening polymer or surfactant.

2. A dosage form coated with the coating of claim 1.

3. The coating of claim 1, wherein said microcrystalline cellulose and carrageenan are present in a ratio of 90:10 to 65:35 wt %, respectively.

4. The coating of claim 1 further containing at least one of a filler, coloring agent, anti-tack agent or preservative.

5. A method of coating a pharmaceutical, nutraceutical or veterinary solid dosage form comprising the steps of hydrating the composition of claim 1 followed by spray coating said hydrated coating composition onto said solid dosage form.

6. The coating of claim 1, wherein said microcrystalline cellulose and carrageenan are present in an amount of 75:25 wt %, respectively.
